(19) United States
(12) Patent Application Publication
(10) Pub. No.: US 2025/0295876 A1
(43) Pub. Date: Sep. 25, 2025

(54) MASK UTILIZING AN OPTICAL FIBER BASED SENSOR

(71) Applicant: KONINKLIJKE PHILIPS N.V., EINDHOVEN (NL)

(72) Inventors: CORNELIS PETRUS HENDRIKS, EINDHOVEN (NL); JOACHIM JOHANNES KAHLERT, AACHEN (DE); DAAN ANTON VAN DEN ENDE, BREDA (NL); JOYCE VAN ZANTEN, WAALRE (NL); JONATHAN SAYER GRASHOW, CHESWICK, PA (US); CORNELIS REINDER RONDA, AACHEN (DE)

(21) Appl. No.: 19/086,663

(22) Filed: Mar. 21, 2025

Related U.S. Application Data
(60) Provisional application No. 63/569,487, filed on Mar. 25, 2024.

(51) Int. Cl. A61M 16/00

(52) U.S. Cl. CPC A61M 16/024; A61M 2205/3306; A61M 2205/332

(57) ABSTRACT

A pressure support system includes a pressure generating device, a patient interface device, an optical fiber having distal end provided on or within the patient interface device, a number of optical fiber-based sensors provided in the distal end, and a light source and an optical spectrum analyzer coupled to the proximal end of the fiber. The light source provides source light to the optical fiber-based sensor(s) and the optical spectrum analyzer receives reflected light from the sensor(s). A controller is configured to receive an output of the analyzer and to (i) determine a measure of a seal, stability, and/or fit of the patient interface device based on the output of the analyzer, (ii) determine a degree of wear of the patient interface device based on the output of the analyzer, and/or (iii) automatically identify a component of the patient interface device based on the output of the analyzer.

### CROSS-REFERENCE TO RELATED APPLICATIONS

This patent application claims the priority benefit under 35 U.S.C. § 119(e) of U.S. Provisional Application No. 63/569,487, filed on Mar. 25, 2024, the contents of which are herein incorporated by reference.

### BACKGROUND OF THE INVENTION

### 1. Field of the Invention

The disclosed concept relates generally to pressure support systems, and, more particularly, to a pressure support system in which an optical fiber based sensor, such as a fiber Bragg grating (FBG), is utilized to automatically identify the mask used in the system and/or measure mask function and/or mask wear out.

### 2. Description of the Related Art

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation. It is also known to deliver continuous positive airway pressure (CPAP) or variable airway pressure, which varies with the patient's respiratory cycle, to treat a medical disorder, such as sleep apnea syndrome, in particular, obstructive sleep apnea (OSA), or congestive heart failure.

Non-invasive ventilation and pressure support therapies involve the placement of a respiratory patient interface device, including a mask component, on the face of a patient. The mask component may be, for example and without limitation, a nasal mask that covers the patient's nose, a nasal cushion having nasal prongs that are received within the patient's nares, a pillow-style nasal cushion that engages the patient's nares without being inserted therein, a nasal/oral mask that covers the patient's nose and mouth, or a full face mask that covers the patient's face. The respiratory patient interface device interfaces a pressure/flow generating device with the airway of the patient so that a flow of breathing gas can be delivered from the pressure/flow generating device to the airway of the patient.

In non-invasive ventilation and pressure support therapies, it can be advantageous to be able to automatically identify the type and/or size of the mask that is used during therapy to: (i) help first-time users to optimally use and adjust the mask for best comfort and seal, (ii) recommend accessories that can be purchased to enable optimal sealing and comfort (e.g., an adhesive foam layer that can be added to the mask for patients that have a beard), (iii) personalize device (e.g., CPAP) settings and link the mask size/type to patient therapy adherence patterns, and/or (iv) collect data for mask design purposes and/or to determine how a specific face/mask combo links to therapy adherence. The latter could drive insights to optimize mask selection algorithms.

A number of prior art on-mask auto-identification methods have been tried with limited success. These methods include, for example, features provided on the mask to enable identification by measuring an electrical resistance, RFID tags containing stored mask identification data that are affixed to the mask, and mask identification using measured pressure and flow features of the mask.

In addition, the effectiveness of therapy and the adherence to therapy can often be driven by factors such as mask comfort, mask seal, mask stability, mask fit, and mask wear out. Current methods for monitoring such factors have proven to be largely ineffective. For example, current resistance-based metal sensors that have been employed in masks for this purpose are typically fragile and have rigid interconnects and wires that are difficult to integrate in a mask. As a result, their presence may actually hinder or even worsen the intended function.

### SUMMARY OF THE INVENTION

In one embodiment, a pressure support system is provided that includes a pressure generating device for generating a flow of breathing gas, a patient interface device for delivering the flow of breathing gas to the airways of a patient, an optical fiber having a proximal end and a distal end, wherein at least the distal end is provided on or within a component of the patient interface device, a number of optical fiber-based sensors provided in the distal end of the optical fiber, and a light source and an optical spectrum analyzer coupled to the proximal end of the optical fiber. The light source is structured and configured to provide source light to the number of optical fiber-based sensors and the optical spectrum analyzer is structured and configured to receive reflected light from the number of optical fiber-based sensors. A controller is also included and is structured and configured to receive an output of the optical spectrum analyzer and to (i) determine a measure of a seal, stability, and/or fit of the component of the patient interface device based on the output of the optical spectrum analyzer, (ii) determine a degree of wear of the component of the patient interface device based on the output of the optical spectrum analyzer, and/or (iii) automatically identify the component of the patient interface device based on the output of the optical spectrum analyzer.

In another embodiment, a method of determining a measure of a seal, stability, and/or fit of a component of a patient interface device, determining a degree of wear of the component of the patient interface device, and/or automatically identifying the component of the patient interface device is provided, wherein at least a distal end of an optical fiber is provided on or within the component of the patient interface device. Also, a number of optical fiber-based sensors are provided in the distal end of the optical fiber, and a light source and an optical spectrum analyzer are coupled to a proximal end of the optical fiber. The method includes providing a source light to the number of optical fiber-based sensors from the light source, receiving in the optical spectrum analyzer reflected light from the number of optical fiber-based sensors, and (i) determining a measure of a seal, stability, and/or fit of the component of the patient interface device based on the output of the optical spectrum analyzer, (ii) determining a degree of wear of the component of the patient interface device based on the output of the optical spectrum analyzer, and/or (iii) automatically identifying the component of the patient interface device based on the output of the optical spectrum analyzer.

In yet another embodiment, a patient circuit for a pressure support system is provided that includes a patient interface device for delivering a flow of breathing gas to the airways of a patient, an optical fiber having a proximal end and a distal end, wherein at least the distal end is provided on or within a component of the patient interface device, and a number of optical fiber-based sensors provided in the distal end of the optical fiber.

### BRIEF DESCRIPTION OF THE DRAWINGS

A full understanding of the invention can be gained from the following description of the preferred embodiments when read in conjunction with the accompanying drawings in which:

FIG. 1 is a schematic diagram of a pressure support system in which an optical fiber based sensor is utilized to automatically identify a mask and/or to measure mask comfort, fit, seal, stability and/or wear out according to an embodiment of the disclosed concept;

FIG. 2 is a schematic diagram illustrating an exemplary fiber Bragg grating (FBG) and the operation thereof that may be employed as part of the disclosed concept;

FIG. 3 is a block diagram of an electronics module forming part of the pressure support system of FIG. 1;

FIG. 4 is a schematic diagram illustrating the operation of the system of FIG. 1 according to an exemplary embodiment;

FIGS. 5 and 6 show schematic diagrams illustrating the positioning of optical fiber based sensors in a mask according to an exemplary embodiment;

FIG. 7 is a schematic diagram of a pressure support system in which an optical fiber based sensor is utilized to automatically identify a mask and/or to measure mask comfort, fit, seal, stability and/or wear out according to an alternative embodiment of the disclosed concept;

FIGS. 8 and 9 are block diagrams of electronics modules forming a part of the system of FIG. 7;

FIG. 10 is a schematic diagram of a pressure support system in which an optical fiber based sensor is utilized to automatically identify a mask and/or to measure mask comfort, fit, seal, stability and/or wear out according to a further alternative embodiment of the disclosed concept;

FIG. 11 is a block diagram of an electronics module forming a part of the system of FIG. 10;

FIG. 12 is a schematic diagram of a pressure support system in which an optical fiber based sensor is utilized to measure the function of a headgear component according to a further alternative embodiment of the disclosed concept;

FIG. 13 is a flow diagram that shows the workflow according to a particular exemplary embodiment of the disclosed concept; and

FIG. 14 illustrates exemplary mask function classifications according to a particular exemplary embodiment of the disclosed concept.

### DETAILED DESCRIPTION OF THE INVENTION

As used herein, the singular form of “a”, “an”, and “the” include plural references unless the context clearly dictates otherwise.

As used herein, the statement that two or more parts or components are “coupled” shall mean that the parts are joined or operate together either directly or indirectly, i.e., through one or more intermediate parts or components, so long as a link occurs.

As used herein, “directly coupled” means that two elements are directly in contact with each other.

As used herein, the term “number” shall mean one or an integer greater than one (i.e., a plurality).

As used herein, the term “controller” shall mean a programmable analog and/or digital device (including an associated memory part or portion) that can store, retrieve, execute and process data (e.g., software routines and/or information used by such routines), including, without limitation, a field programmable gate array (FPGA), a complex programmable logic device (CPLD), a programmable system on a chip (PSOC), an application specific integrated circuit (ASIC), a microprocessor, a microcontroller, a programmable logic controller, or any other suitable processing device or apparatus. The memory portion can be any one or more of a variety of types of internal and/or external storage media such as, without limitation, RAM, ROM, EPROM(s), EEPROM(s), FLASH, and the like that provide a storage register, i.e., a non-transitory machine readable medium, for data and program code storage such as in the fashion of an internal storage area of a computer, and can be volatile memory or nonvolatile memory.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

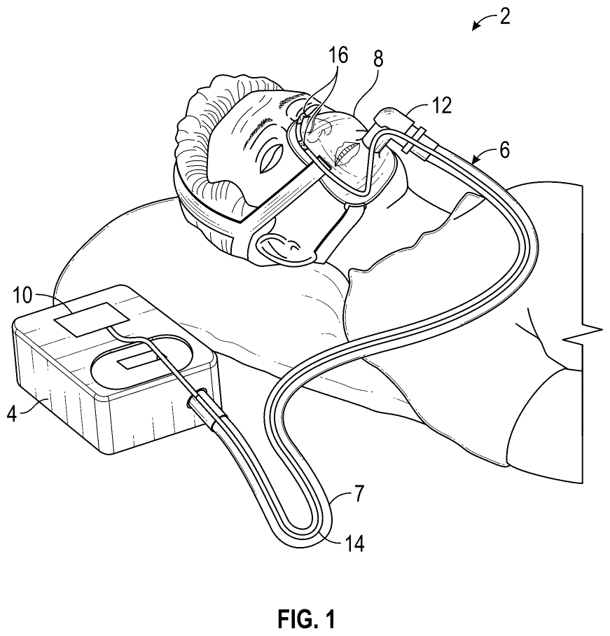

FIG. 1 is a schematic diagram of a pressure support system 2 in which an optical fiber based sensor is provided as part of a patient interface device and is utilized to automatically identify a mask used in pressure support system 2 and/or to measure mask comfort, fit, seal, stability and/or wear out according to an embodiment of the disclosed concept. In the exemplary embodiment, the optical fiber based sensor is a fiber Bragg grating (FBG). It will be understood, however, that this is meant to be exemplary only and that other types of optical fiber based sensors may be employed in pressure support system 2.

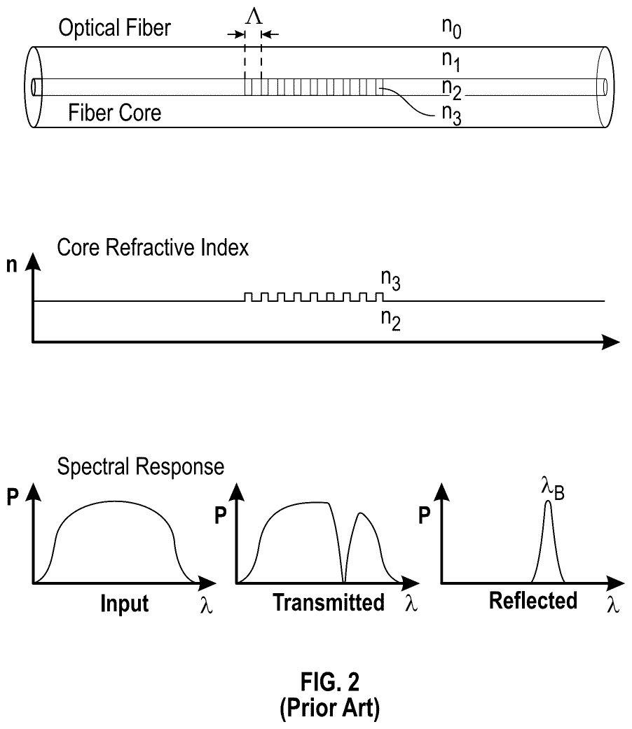

As is known in the art, an FBG is a type of sensor that is constructed in a short segment of optical fiber. More specifically, an FBG is created by periodically modulating the refractive index in a short section of the core of an optical fiber core. An optical source, such as a laser source, at the proximal end of the fiber containing the FBG provides a wide spectrum light to the fiber/FBG, and in response the spectrum reflected by the FBG (known as the reflection spectrum) appears as a narrow sharp peak. This is illustrated in FIG. 2. The reflected peak of the FBG will shift in response to perturbations (i.e., changes in the periodic modulation) that affect the FBG. Such perturbations can be a strain, a pressure change, a shape change (bending), or a temperature change, among others. The shift of the reflection spectrum can be measured with an optical spectrum analyzer and can be used to quantify the perturbation. As such, an FBG can be used as a strain, pressure, force, temperature, or shape sensor. Multiplexing of FBGs is straightforward by encoding each FBG sensor with a unique wavelength along a single fiber. Until recently, a disadvantage of FBG sensors has been the cost of the readout system, especially for very precise and high-resolution applications. More recently, however, ultra-low-cost solutions have been developed using smartphone components.

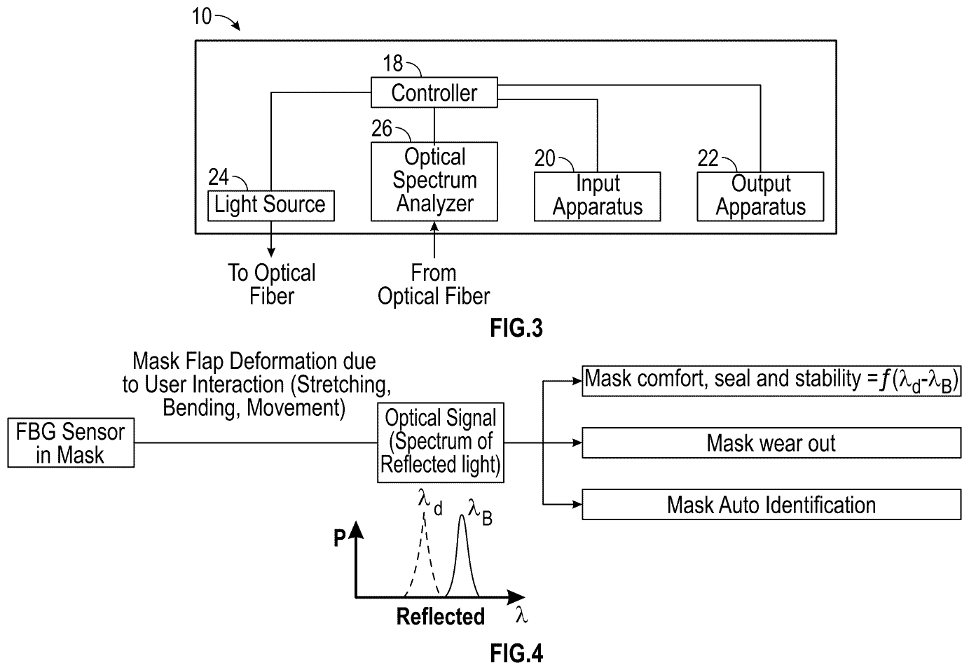

Referring again to FIG. 1, pressure support system 2 is adapted to provide a regimen of respiratory therapy to a patient. Pressure support system 2 includes a pressure generating device 4 and a patient circuit 6 including a delivery conduit 7 and a patient interface device 8. Pressure generating device 4 is structured to generate a flow of breathing gas and may include, without limitation, ventilators, constant pressure support devices (such as a continuous positive airway pressure device, or CPAP device), variable pressure devices (e.g., BiPAP®, Bi-Flex®, or C-Flex™ devices manufactured and distributed by Koninklijke Philips N.V.), and auto-titration pressure support devices. As seen in FIG. 1, pressure generating device 4 includes an electronics module 10, which is described in greater detail herein in connection with FIG. 3.

Delivery conduit 7 is structured to communicate the flow of breathing gas from pressure generating device 4 to patient interface device 8. Typically, delivery conduit 7 includes one or more individual conduits or tubes, a first end of which couples with pressure generating device 4 and a second end of which couples with patient interface device 8. In the illustrated embodiment, the second end is coupled with patient interface device 8 through a fluid coupling device 12 (e.g., an elbow conduit) of patient interface device 8.

In the exemplary embodiment illustrated in FIG. 1, patient interface device 8 is a nasal/oral mask structured to be placed on the face of a patient. Any type of patient interface device 8, however, which facilitates the delivery of the flow of breathing gas to, and the removal of a flow of exhalation gas from, the airway of such a patient may be used while remaining within the scope of the disclosed concept. In the embodiment shown in FIG. 1, patient interface device 8 includes a flexible cushion, a rigid shell or faceplate, and a headgear component to secure patient interface device 8 to the patient's head. An opening in the shell of patient interface device 8, to which fluid coupling device 12 is coupled, allows the flow of breathing gas from pressure generating device 4 to be communicated to an interior space defined by the shell and cushion of patient interface device 8, and then to the airway of a patient. The opening in the shell also allows the flow of exhalation gas from the airway of the patient to be communicated to an exhaust port assembly in fluid coupling device 12.

As seen in FIG. 1, pressure support system 2 includes an optical fiber 14 that extends through patient circuit 6. In particular, optical fiber 14 has a proximal end that is coupled to electronics module 10, a middle portion that is provided on or within delivery conduit 7 (or a separate lumen), and a distal end that is provided on and/or within the mask of patient interface device 8. In the exemplary embodiment, optical fiber 14 enters the mask at the location of fluid coupling device 12 with a fiber coupler and/or with some slack if needed to accommodate rotations of a swivel-type connector. A number of FBGs 16 are provided in the distal end of optical fiber 14. The optical fiber 14 and FBGs 16 are integrated in the mask (e.g., in the flexible cushion thereof) in such a way that FBGs 16 are in critical locations, such as on the nose bridge, near the eyes, cheek, etc. Each FBG 16 may have its own characteristic wavelength to allow multiplexing. Although FBG sensors are stable, long-term storage may influence the sensor characteristics in applications which require a high accuracy. This can be mitigated by calibrating the sensor response (and creating a calibration file) after storage, but before usage.

FIG. 3 is a block diagram of electronics module 10 according to an exemplary embodiment of the disclosed concept. Electronics module 10 includes a controller 18, an input apparatus 20 (such as a keyboard), and an output apparatus 22 (such as a liquid crystal display). A user is able to provide input into controller 18 using input apparatus 20, and controller 18 provides output signals to output apparatus 22 to enable output apparatus 22 to display information to the user as described herein. Electronics module 10 further includes a light source 24 and an optical spectrum analyzer 26 (having an internal photodetector). Light source 24 is structured and configured to provide a wide spectrum light to optical fiber 14 and FBGs 16, and optical spectrum analyzer is structured and configured to receive the reflection spectrum of each FBG 16 and to identify the wavelength of each reflected peak. In the exemplary embodiment, both light source 24 and optical spectrum analyzer 26 are miniature semiconductor devices that are capable of being mounted on a printed circuit board within a relatively small form factor. In one particular embodiment, light source 24 is a laser diode and optical spectrum analyzer 26 is a spectrum analyzer integrated circuit device.

The memory portion of controller 18 has stored therein a number of routines (comprising computer executable instructions) that are executable by the processor portion of controller 18, including routines for implementing the disclosed concept as described herein for automatically identifying the mask used in pressure support system 2 and/or for measuring mask function (comfort, seal, stability and/or fit) and/or mask wear out. This functionality is shown schematically in FIG. 4. Specifically, if there is no perturbation of FBGs 16, the reflected wavelength is λB. If there is a perturbation due to, for example, mask flap deformation, the reflected wavelength is λd. The mask comfort, seal, and stability can be analyzed based on the wavelength shift λd−λB. Mask wear out and automatic mask identification can also be based on the wavelength shift λd−λB.

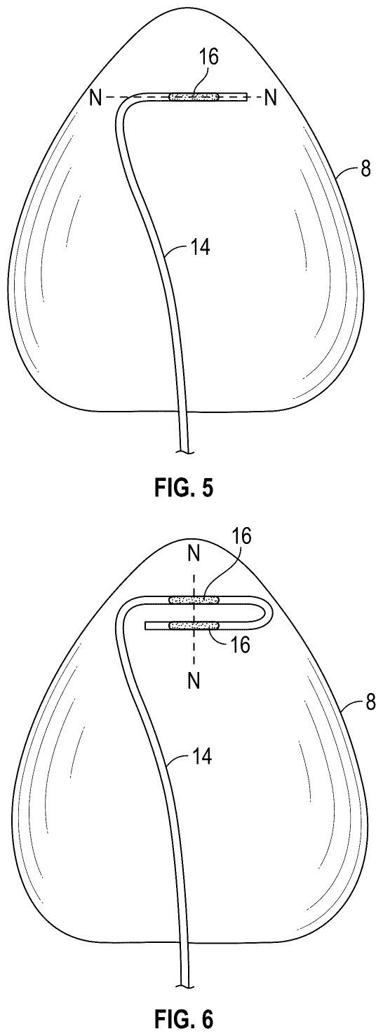

Coupling challenges, i.e., an FBG 16 responding to multiple simultaneous stimuli, can be solved with basic mechanics (theoretical principles), or can be ignored. In one particular embodiment, a number of options are possible to detect and separate mask cushion flap bending and stretching as follows. In a first option, loose integration of the fiber in a channel or lumen formed in or over molded on the mask may be used to measure bending only, wherein λd is a function of bending radius (λd=f (bending radius)). In a second option, shown in FIG. 5, an FBG 16 may be positioned in a neutral line (N-N) of the mask to measure stretch only, λd=f (stretch). This second option assumes that bending in the neutral line does not change the pitch of the grating (no elongation or compression) and therefore does not cause a wavelength shift. In a third option, shown in FIG. 5, two FBGs 16 are positioned on both sides of the neutral line, for example in a configuration as shown in FIG. 6, to simultaneously measure stretching and bending. In this third option, when λd,1=−λd,2, the mask/seal is bending only; when |λd,1|≠|λd,2|, the mask/seal is bending and stretching; when λd,1=λd,2, the mask/seal is stretching only.

In a further alternative embodiment, one or more of the FBGs 16 may be a chirped FBG. In a chirped FBG, there is a linear variation of the FBG period along the grating length. In this alternative, when the FBG is stretched, the whole spectrum is shifted; when the FBG is bent, a part of the spectrum is shifted.

In normal seal deformation, the FBG sensor compression and response due to a radial pressure is likely much smaller than the sensor deformation due to bending or stretching. As a result, the radial pressure contribution can most likely be ignored in connection with the disclosed concept. This can be confirmed through calibration.

In addition, although an FBG can be used as a temperature sensor, it is believed that the temperature sensitivity of FBG sensors will not be a problem for implementation of the disclosed concept. In other words, sensor output variations due to thermal interaction with the human skin are not believed to be problematic for implementation of the disclosed concept. Under normal conditions, the skin surface temperature varies between 33-37° C., while the temperature sensitivity is only 24 pm/° C., which is a small number compared to the nm-level variations in deformation under strain. Additionally, postprocessing technologies may be employed to isolate strain and temperature response, so an FBG can be used as a strain and temperature sensor simultaneously. Furthermore, FBG cooling due to leak flow can likely be ignored if the air is heated (37° C.) and humidified (100% RH), in which case there is no cooling effect due to evaporation or convection. Rather, it could actually be helpful to detect leak locations based on temperature.

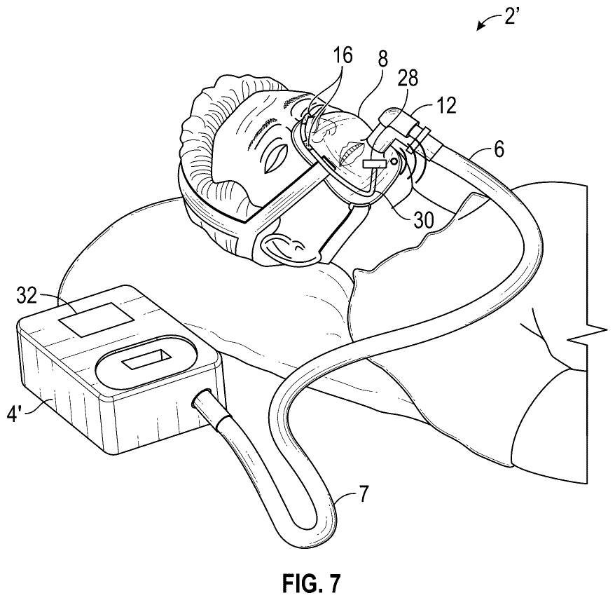

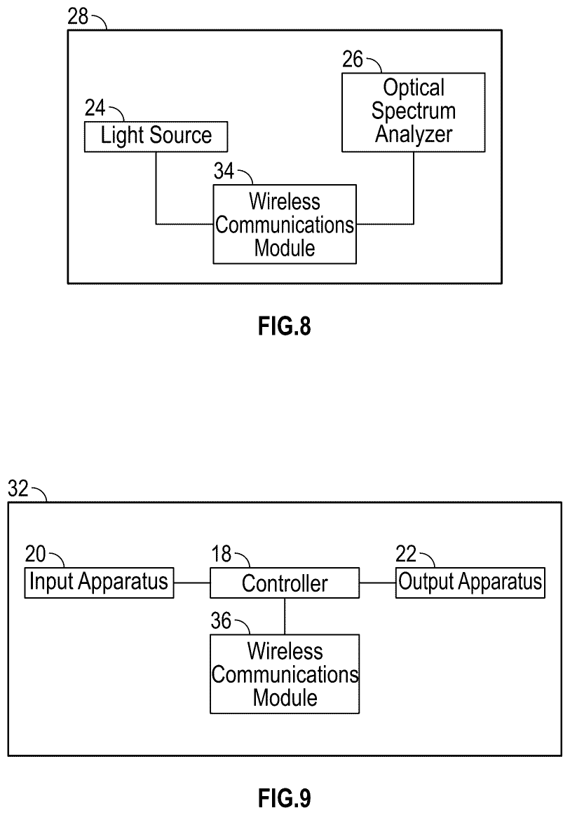

FIG. 7 is a schematic diagram of a pressure support system 2′ in which an optical fiber based sensor is provided as part of a patient interface device and is utilized to automatically identify a mask used in pressure support system 2 and/or to measure mask comfort, fit, seal, stability and/or wear out according to an alternative embodiment of the disclosed concept. Pressure support system 2′ is similar to pressure support system 2, and like parts are labelled with like reference numerals. In this embodiment, however, patient interface device 8 includes optical fiber 30 having one or more FBGs 16 mounted therein or thereon as described elsewhere herein. In addition, an electronics module 28 is provided as part of patient interface device 8 and is mounted in or on the mask portion thereof. Electronics module 28 is shown in FIG. 8 and described below. This embodiment includes an alternative pressure generating device 4′ that is similar to pressure generating device 4, except that it includes an electronics module 32 as shown FIG. 9.

Referring to FIG. 8, electronics module 28 includes a light source 24 and an optical spectrum analyzer 26 as described elsewhere herein. In addition, electronics module 28 includes a wireless communications module 34, such as, without limitation, a Wi-Fi or a Bluetooth module, for enabling wireless communications to and from electronics module 28. Referring to FIG. 9, electronics module 32 includes a controller 18, an input apparatus 20, and an output apparatus 22 as described elsewhere herein. In addition, electronics module 32 includes a wireless communications module 36 that is compatible with wireless communications module 34. As a result, electronics module 28 and electronics module 32 are able to wirelessly communicate with one another.

Operation of pressure support system 2′ is similar to operation of pressure support system 2, except that in pressure support system 2′, the wide spectrum light is provided to FBGs 16 through optical fiber 30 by light source 24 of electronics module 28, and the reflected peak is received by optical spectrum analyzer 26 of electronics module 28. That information is then wirelessly communicated to controller 18 of electronics module 32 for processing thereby as described elsewhere herein in order to measure mask function and/or auto identify the mask used in pressure support system 2′.

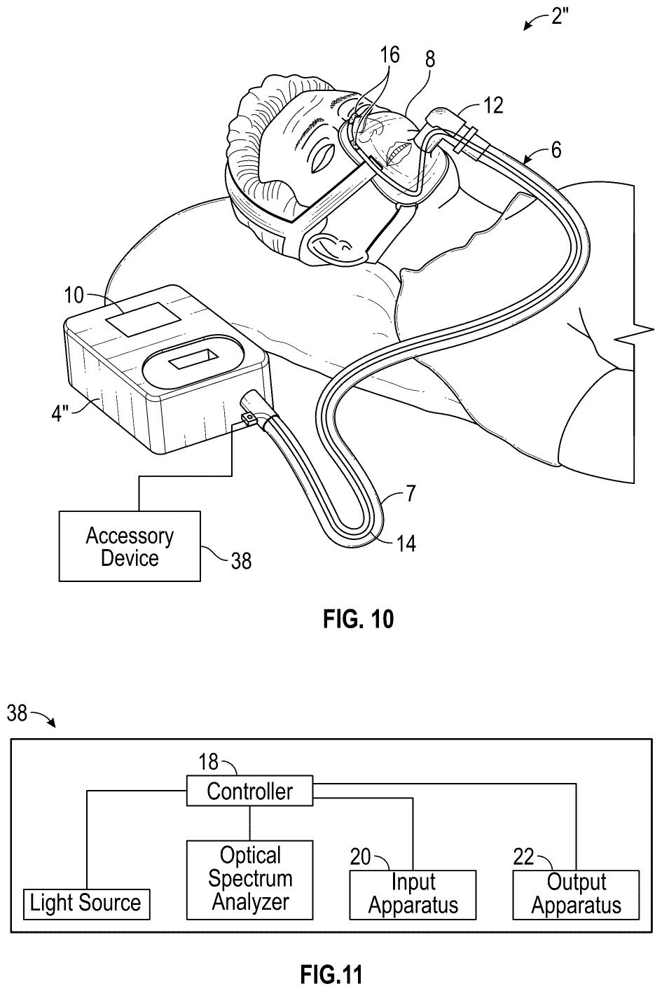

FIG. 10 is a schematic diagram of a pressure support system 2″ in which an optical fiber based sensor is provided as part of a patient interface device and is utilized to automatically identify a mask used in pressure support system 2″ and/or to measure mask comfort, fit, seal, stability and/or wear out according to another alternative embodiment of the disclosed concept. Pressure support system 2″ is similar to pressure support system 2, and like parts are labelled with like reference numerals. In this embodiment, however, pressure support system 2″ includes an accessory tool, such as, without limitation, a mask selector tablet or other computing device, which is shown schematically in FIG. 11. As seen in FIG. 11, accessory device 38 includes a controller 18, an input apparatus 20, an output apparatus 22, a light source 24, and an optical spectrum analyzer 26, all as described elsewhere herein. Thus, in this embodiment, the functionality that was previously provided by those components as part of pressure generating device 4 is now provided by accessory device 38. Otherwise, processing of the signals to measure mask function and/or auto identify a mask are as the same as described in connection with pressure support system 2 as described herein.

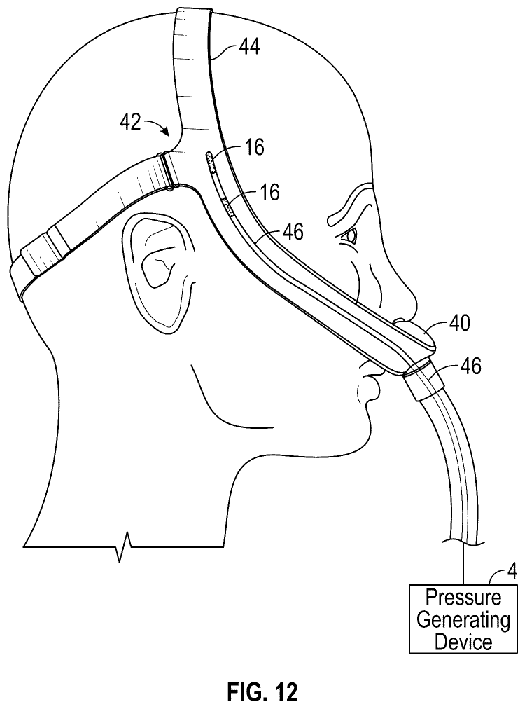

FIG. 12 provides still a further alternative embodiment which is similar to pressure generating pressure support system 2 except that in this embodiment the optical fiber 14 is provided on or within the headgear portion of a patient interface device for detecting the function (comfort, fit, stability) of the headgear. In this embodiment, the patient interface device comprises a pillows-style nasal cushion 40 that is coupled to a headgear component 42 having a rigid frame member 44. An optical fiber 46 having a number of FBGs 16 is provided on or within rigid frame member 44 of headgear component 42. In this embodiment, FBGs 16 are used to sense strain/bending in frame member 44 to detect over/under-tightening of headgear component 42, cheek contact by headgear component 42, or other headgear function (fit, comfort, stability).

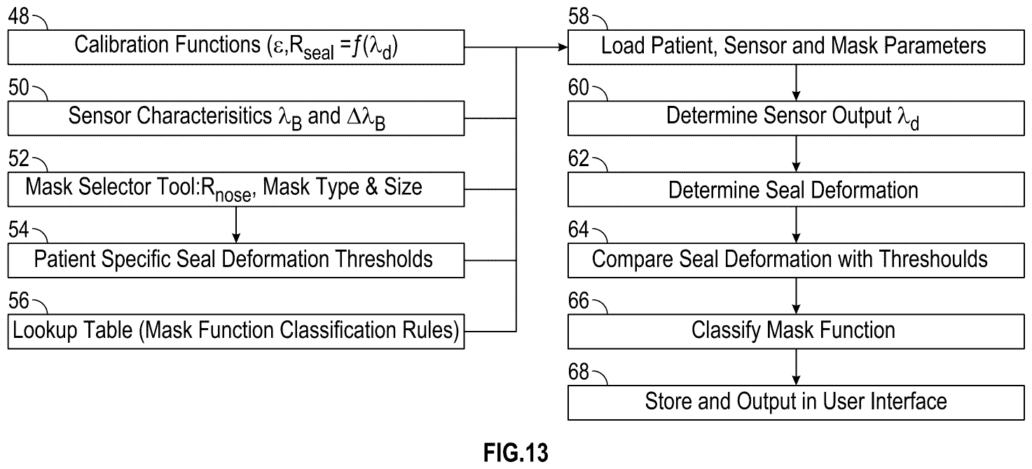

As discussed herein, FBG sensors respond to perturbation. They do not directly measure mask function parameters such as a skin pressure or a leak flow. So, as described herein, the sensed perturbation needs to be translated to a mask function parameter (e.g., comfort, seal, stability), including a classification of the mask function (e.g., good, bad) in the exemplary embodiment. This translation and classification depend on factors such as the sensor (FBG) location (for example, FBG stretching on the nose bridge indicates a skin pressure while at another location FBG stretching indicates a leak), mask type and size, and patient facial geometry. One particular exemplary embodiment of the disclosed concept is described below. This embodiment describes how to evaluate and characterize an exemplary full-face mask that rests on the nose bridge of a patient using an algorithm which takes as input FBG sensor data (e.g., one or two sensors), patient data from a source such as a mask selector tool (e.g., implemented by accessory device 38), and the mask type and size. FIG. 13 is a flow diagram that shows the workflow (i.e., algorithm) according to this particular exemplary embodiment.

Referring to FIG. 13, the sensor, patient, and mask parameters which are needed inputs for the function evaluation are obtained from a number of sources (e.g., via an input apparatus 20 or from an accessory device such as accessory device 38) and stored in the memory of the device that is executing the algorithm (e.g. controller 18 as described herein). That information includes: (i) a number of sensor calibration functions 48 obtained in research and development (R&D), including a function for mask seal bending radius, Rseal=f(λd), and a function for mask seal stretch, ε=f(λd); (ii) sensor characteristics 50 including the Bragg grating wavelength λB and the noise level ΔλB of the FBGs 16 coupled to the mask in question that are obtained in R&D or during home setup in a reference situation where the mask is new, off and unloaded (not lying on a nightstand, for example); (iii) mask parameters 52 including the patient nose radius Rnose and the mask type and size from, for example, a mask selector tool or by manual input, and (iv) patient specific thresholds 54 for mask seal deformation. In the exemplary embodiment, the patient specific thresholds 54 includes a stretch threshold (relating to skin pressure/discomfort), a bending threshold (relating to degree of sealing), and a movement threshold (relating to mask stability).

In the exemplary embodiment, the stretch threshold is determined using a lookup table 56 that takes as input the nose bridge radius Rnose and generates as output the stretch threshold as the maximum stretch εmax at which the skin normal pressure stays below the capillary closing pressure which is 3 kPa. The lookup table may be created in research and development using finite element modeling to evaluate the skin pressure for a range of nose radii, seal geometries and seal stretches. In the exemplary embodiment, the bending threshold is determined based on the assumption that when Rseal (the actual radius of the mask when work)<Rnose, there is a risk for leakage. Thus, in this embodiment, the bending threshold value is Rnose. Optionally, a patient specific finite element analysis of the mechanical mask-face interaction can reveal FBG sensor deformation corresponding to discomfort and leakage, including the quantification of pressures and gaps. Such an analysis can be implemented for example as a future service or feature in a mask selector tool. With respect to the movement threshold, mask movement likely occurs when the variation in the reflected wavelength of an FGB 16 is larger than the noise level in the baseline situation (Δλd>ΔλB), and when the frequency of the wavelength variation Δλd is smaller than the frequency of physiological signals (respiration rate, heart rate). Alternatively, it could also be possible to create mask-specific thresholds that apply to all patients. For example, if a mask cushion deforms beyond a certain range, then the cushion is exerting an unacceptable pressure on the face.

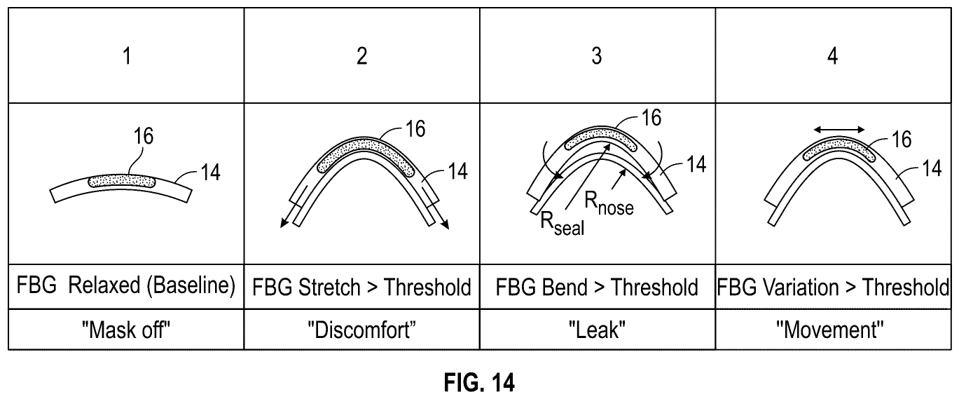

As seen in FIG. 13, these parameters are loaded at step 58. Then, at step 60, the mask is donned by the patient and the sensor (FBG 16) output is acquired and processed. The reflected wavelength is λ=λd. Next, at step 62, local seal deformation values for seal, bending, and movement are obtained using the sensor calibration functions. The method then proceeds to step 64, wherein the measured seal deformation values (stretch, bend, movement) are compared with the deformation thresholds. This can be implemented in the form of a simple test (seal deformation>threshold) generating as output true or false or a digit. Next, at step 66, the mask functions are evaluated using a lookup table or rules which take as input the local seal deformation (from step 62) and the threshold values 54 (and the comparison from step 64). At step 68, the method generates as outputs the mask function classifications as shown in FIG. 14, for example. The classifications may be in the form of risk indicators (HIGH/LOW), or a digit which triggers an indication or a warning in the user interface (0=not used, 1=OK, 2=red flag), or qualitative descriptions (“leak”). At step 68, the results are recorded and communicated in a user interface such as output apparatus 22.

In further alternative exemplary embodiments, an FBG sensor array comprising multiple FBGs 16 can be distributed over the whole mask seal perimeter in a high-resolution array. This is especially useful for detecting local buckling of the seal, which leads to high leakage. FBGs 16 are good at detecting buckling as the local deformation is very high and the high-resolution array allows for precise localization of the buckling. This can be used as troubleshooting data for clinicians or DMEs to help them evaluate the leakage source and propose a better fitting mask.

In another exemplary embodiment, mask wear-out detection is characterized by a gradual wavelength shift over time of the unloaded mask, compared to a new unloaded mask (locally the seal slowly elongates due to creep deformation, due to which the seal starts to buckle). This embodiment enables the possibility of predicting the time that a mask still can be used and when a new mask is needed, for example, by using thresholds such as a creep >10% or 50%. This embodiment would also enable new masks to be ordered just in time.

In another exemplary embodiment, mask type auto identification is revealed by a characteristic wavelength at zero hour (when the mask is new and unloaded, or by a differentiating wavelength in a non-deforming part of the fiber—for example in the mask frame). This could also be used to prevent counterfeiting.

In still another exemplary embodiment, FBG sensors as described herein combined with actuating headgear may be used to adjust the skin pressure, seal, and stability in real time.

While specific embodiments of the invention have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of disclosed concept which is to be given the full breadth of the claims appended and any and all equivalents thereof.

What is claimed is:

1. A pressure support system, comprising:
a pressure generating device for generating a flow of breathing gas;
a patient interface device for delivering the flow of breathing gas to the airways of a patient;
an optical fiber having a proximal end and a distal end, wherein at least the distal end is provided on or within a component of the patient interface device;
a number of optical fiber-based sensors provided in the distal end of the optical fiber;
a light source and an optical spectrum analyzer coupled to the proximal end of the optical fiber, the light source being structured and configured to provide source light to the number of optical fiber-based sensors and the optical spectrum analyzer being structured and configured to receive reflected light from the number of optical fiber-based sensors; and
a controller structured and configured to receive an output of the optical spectrum analyzer and to (i) determine a measure of a seal, stability, and/or fit of the component of the patient interface device based on the output of the optical spectrum analyzer, (ii) determine a degree of wear of the component of the patient interface device based on the output of the optical spectrum analyzer, and/or (iii) automatically identify the component of the patient interface device based on the output of the optical spectrum analyzer.

2. The pressure support system according to claim 1, wherein the number of optical fiber-based sensors are a number of FBGs, wherein the source light is a wide spectrum light, wherein the reflected light is a reflection spectrum of each of the FBGs, and wherein the output of the optical spectrum analyzer is a wavelength of each reflected peak.

3. The pressure support system according to claim 1, wherein the component of the patient interface device is a mask or a headgear.

4. The pressure support system according to claim 1, wherein the patient interface device is coupled to the pressure generating device by a delivery conduit, and wherein a portion of the optical fiber is provided on or within the delivery conduit.

5. The pressure support system according to claim 4, wherein the light source, the optical spectrum analyzer, the proximal end of the optical fiber and the controller are provided within the pressure generating device.

6. The pressure support system according to claim 4, wherein the light source, the optical spectrum analyzer, and the controller are provided within an accessory device coupled to the optical fiber.

7. The pressure support system according to claim 1, wherein the light source, the optical spectrum analyzer, and the proximal end of the optical fiber are provided on or within the patient interface device, further comprising a wireless communication module provided on or within the patient interface device for allowing the output of the optical spectrum analyzer to be wirelessly transmitted to the controller.

8. The pressure support system according to claim 2, wherein the controller structured and configured to determine a deformation value as a function of the reflection spectrum of each of the FBGs, compare the deformation value to a threshold, and evaluate the seal, the stability, and/or the fit of the component based on the comparison of the deformation value to a threshold.

9. The pressure support system according to claim 2, wherein the controller is structured and configured to determine the degree of wear of the component of the patient based on a shift over time of the reflection spectrum of each of the FBGs.

10. A method of determining a measure of a seal, stability, and/or fit of a component of a patient interface device, determining a degree of wear of the component of the patient interface device, and/or automatically identifying the component of the patient interface device, wherein at least a distal end of an optical fiber is provided on or within the component of the patient interface device, wherein a number of optical fiber-based sensors are provided in the distal end of the optical fiber, and wherein a light source and an optical spectrum analyzer are coupled to a proximal end of the optical fiber, the method comprising:
providing a source light to the number of optical fiber-based sensors from the light source;
receiving in the optical spectrum analyzer reflected light from the number of optical fiber-based sensors; and
(i) determining a measure of a seal, stability, and/or fit of the component of the patient interface device based on the output of the optical spectrum analyzer, (ii) determining a degree of wear of the component of the patient interface device based on the output of the optical spectrum analyzer, and/or (iii) automatically identifying the component of the patient interface device based on the output of the optical spectrum analyzer.

11. The method according to claim 10, wherein the number of optical fiber-based sensors are a number of FBGs, wherein the source light is a wide spectrum light, wherein the reflected light is a reflection spectrum of each of the FBGs, and wherein the output of the optical spectrum analyzer is a wavelength of each reflected peak.

12. The method according to claim 10, wherein the component of the patient interface device is a mask or a headgear.

13. The method according to claim 11, including determining a deformation value as a function of the reflection spectrum of each of the FBGs, compare the deformation value to a threshold, and evaluate the seal, the stability, and/or the fit of the component based on the comparison of the deformation value to a threshold.

14. The method according to claim 11, including determining the degree of wear of the component of the patient based on a shift over time of the reflection spectrum of each of the FBGs.

15. A patient circuit for a pressure support system, comprising:
a patient interface device for delivering a flow of breathing gas to the airways of a patient;
an optical fiber having a proximal end and a distal end, wherein at least the distal end is provided on or within a component of the patient interface device; and
a number of optical fiber-based sensors provided in the distal end of the optical fiber.

16. The patient circuit according to claim 15, wherein the component of the patient interface device is a mask of a headgear.

17. The patient circuit according to claim 15, further comprising a delivery conduit coupled to the patient interface device, wherein a portion of the optical fiber is provided on or within the delivery conduit.

18. The patient circuit according to claim 15, wherein a light source, an optical spectrum analyzer, a proximal end of the optical fiber and a wireless communication module are provided on or within the patient interface device.
